(19) United States
(12) Patent Application Publication
(10) Pub. No.: US 2010/0241207 A1
(43) Pub. Date: Sep. 23, 2010

(54) Implantable Medical Cuff with Electrode Array

(76) Inventor: Henry Bluger, Victoria (CA)

Correspondence Address: Sunstein Kann Murphy & Timbers LLP, 125 SUMMER STREET, BOSTON, MA, 02110-1618, US

(21) Appl. No.: 12/377,544

(22) PCT Filed: Aug. 14, 2007
(86) PCT No.: PCT/CA07/01401

§ 371 Date: Feb. 13, 2009

Related U.S. Application Data
(60) Provisional application No. 60/822,302, filed on Aug. 14, 2006.

(51) Int. Cl. A61N 1/05

(52) U.S. Cl. 607/118

(57) ABSTRACT

An implantable medical assembly comprising at least one electrode carried on an inside surface of a web comprising a biocompatible film wound into a generally tubular configuration, the film containing a plurality of distinct apertures distributed throughout the web to increase its flexibility and to create a means by which biological fluids can penetrate the assembly. The assembly may further comprise at least one wire connected to the electrode to provide a stimulation signal.

### CROSS REFERENCE TO RELATED APPLICATIONS

This application is related to U.S. patent application Ser. No. 10/348,970 filed Jan. 23, 2003 entitled “Implantable Medical Assembly”, which is incorporated herein by reference.

This application is related to U.S. patent application Ser. No. 10/623,639 Jul. 22, 2003 entitled “Implantable Electrical Cable and Methods of Making”, which is also incorporated herein by reference.

### FIELD OF THE INVENTION

The present invention relates to the biomedical arts and in particular to implantable electrode arrays. More particularly, this invention relates to an implantable medical cuff with an electrode array which is to be applied to body tissue to provide an effective electrical connection therewith, for sensing or stimulating purposes.

### BACKGROUND OF THE INVENTION

This invention relates to cuffs for surgical implantation in animals and humans. Cuffs according to the invention have particular application as nerve cuffs and can be made electrically insulating and equipped with electrodes to electrically stimulate or record electrical activity in tissues surrounded by the cuff. The present invention finds particular application in conjunction with cuff electrodes which curl around and snugly engage a nerve trunk, and will be described with particular reference thereto.

It is to be appreciated, however, that the invention is also applicable to other types of implanted biomedical devices for introducing, monitoring, or removing matter or energy by engagement with body tissue.

Cuffs are used in biomedical research and in clinical applications to surround and enclose internal body tissues, such as nerves, arteries, veins, muscles, tendons, ligaments, the oesophagus, intestines, fallopian tubes and other generally tubular internal organs. The functions of cuffs can include: chemically and/or electrically isolating selected tissues from surrounding tissues; supporting electrodes for electrically interacting with tissues inside the cuff; supporting tissues; administering drugs or chemicals to tissues within the cuff; and serving as a platform for physiological experiments. A simple form of cuff that has been widely used in the prior art comprises a section of elastic tube that is slit longitudinally. These cuffs are implanted by separating the edges of the slit to expose the interior of the cuff, placing selected tissues on the cuff and then allowing the cuff to close around the selected tissues. The cuff is then tightly closed and sealed by tying sutures around the cuff at several places along its length. Cuffs of this nature are described in Neuromethods, Vol. 15: Neurophysiological Techniques: Applications to Neural Systems, A. A. Boulton, G. B. Baker and C. H. Vanderwolf, editors. The Humana Press, pp. 65-145, 1990.

Electrical activation of the nervous system has been shown in recent years to offer great hope in restoring some degree of lost sensory and motor function in stroke victims and individuals with spinal cord lesions. Ways in which electrical activation of the nervous system can be utilized to restore a particular function include: (1) the use of surface electrodes to activate the nerves in the general region of interest; (2) the use of intramuscular electrodes, also to activate the nerves in a general region; and (3) the use of nerve cuff electrodes placed around specific nerves of interest and used to activate those nerves specifically and singularly. Direct stimulation of muscles requires a number of electrodes distributed on the muscle, and can consume relatively large amounts of power. In addition, complicated control equipment is required for surveillance of the electrodes to achieve the desired movement of the muscle.

The third alternative offers advantages over the first two in that it requires the lowest levels of stimulating current and hence a minimal amount of charge injected into the tissue. In addition, it allows easy excitation of entire muscles rather than merely parts of muscles, a common situation for the first two categories. Because the use of nerve cuff electrodes requires delicate surgery, they are usually contemplated only when excitation of specific, isolated muscles is desired or when the generation of unidirectional action potentials is required.

Prior art cuffs have been equipped with electrodes and used for interfacing with the nervous system by recording from or stimulating neural tissues. For example, implanted nerve cuffs have been used to record nerve signals from peripheral nerves in animals in a wide range of experimental conditions. Nerve cuff electrodes have been used in stimulation systems with the goal of providing partial voluntary control of muscles that have been paralyzed as a result of lesions caused by spinal cord injury, stroke, or other central neurological disorders. In some cases, partial motor function can be restored by stimulating motor neurons or muscles below the level of the lesion.

Stimulation of nerves can be carried out by placing electrodes locally around the nerves. The controlling equipment and the power supply are generally placed externally on the human body and may be connected to the electrodes by using wires passing through the skin. For obvious reasons, this is not an attractive solution. Alternatively, a transmitter may be placed on the body and a receiver implanted in the body may be wired to the electrodes, nerves being stimulated by the transmitter's transmissions of signals and energy through the skin and flesh to the receiver.

To provide an electrical connection to the peripheral nerve which controls the muscles of interest, an electrode (and sometimes an array of multiple electrodes) is secured to and around the nerve bundle. A wire or cable from the electrode is in turn connected to an implanted package of pulse generation circuitry. The present invention is directed to an improvement in this type of electrode.

One prior art cuff electrode includes a cylinder of dielectric material defining a bore therethrough of sufficient diameter to receive the nerve trunk to be electrically stimulated. The cylinder has a longitudinal split or opening to facilitate spreading the cuff open in order to receive a nerve within the bore. After installation, the longitudinal split is sutured or otherwise held closed. Although suturing holds the cuff in place, an electric current path is defined through the split, which permits current leakage. Two or three annular electrodes are positioned on the inner surface of the bore for use in applying the electrical stimuli. The electric stimuli may be used to generate propagating nerve impulses or may be used to block naturally occurring nerve pulses traveling along the nerve trunk, or to perform similar functions.

A widely used prior-art electrode assembly is formed from a tube of silicone rubber with one or more electrodes secured on the inner surface of the tube. An end-to-end slit is cut through the tube sidewall so the tube can be opened and fitted over the nerve bundle. When so installed, the resiliency of the tube causes it to surround the nerve bundle to urge the electrode against the surface of the tissue. The tube may also be provided with suture flaps for additional anchorage about the nerve bundle. Due to its construction, this style of assembly is usually called a “cuff” electrode.

According to animal-implant studies, traditional cuff electrodes can cause neural damage, and are not wholly satisfactory for long-term implantation. The probable causes of these problems can be summarized as follows:
A. Although it must have some radial flexibility to enable installation over the nerve, the prior-art silicone-rubber tube or sleeve must also be relatively stiff to ensure that the restoring force of the resilient material will position the electrode against the nerve surface, thereby ensuring adequate electrical contact. Excessive gripping and compression of the nerve by the cuff can cause nerve damage by decreasing blood and axoplasmic flow, and by constricting nerve fibers with resulting loss of function. This problem is accentuated by temporary swelling of the nerve caused by the trauma of surgical implantation of the electrode.
B. If a cuff electrode is loosely fitted to limit pressure atrophy of the nerve, a poor electrical contact is made, and this contact is further degraded in time by ingrowth of connective tissue between the cuff and nerve. This ingrowth is sometimes sufficiently marked to lead to compression damage to the nerve as discussed above, or it may cause complete separation of the cuff and nerve.
C. The nerve is encased within the full length of the cuff, blocking a is normal metabolic exchange between the nerve and surrounding tissue. That is, a normal and desired fluid interchange between the nerve and its surrounding environment is prevented or sharply decreased over the length of the cuff.
D. In addition to compression damage, mechanical trauma to the nerve can be caused by torque or bending forces applied by the cuff and its relatively stiff cable during muscle and body movement. These forces may even displace the nerve bundle out of the cuff.
E. Conventional cuff assemblies use electrodes of small surface area, and the resulting high density of electrical charge at the electrode-nerve interface can result in an undesired electrochemical deposition of electrode material on the nerve sheath.

Modern electrical therapeutic and diagnostic devices, such as pacemakers or nerve stimulators for example, require a reliable electrical connection between the device and targeted tissues within the body. In cases of nerve stimulators, in particular, chronically reliable electrical connections have been difficult to attain. In a chronic setting, it has been found that many medical electrical leads may damage nerve tissues either mechanically or electrically or both, as discussed above.

At the same time, supplies of body fluids including blood must be maintained to ensure proper nutrition of the nerve tissue. Blood and lymph are the systems responsible for delivering essential elements and removing harmful wastes from all of the body's tissues. Without blood, the human body would stop functioning. It is thus critical for the health and survival of the target nerve tissues to ensure a free and uninhibited supply of body fluids thereto.

It is therefore an object of the present invention to provide an implantable medical cuff with an electrode array for transmitting and receiving signals to and from the body.

It is a further object of the present invention to provide an improved implantable medical cuff with an electrode array which is capable of providing proper nutrition to the tissue with which it is contact.

It is another object of the present invention to provide an improved implantable medical cuff with an electrode array, which will not cause neural damage, and which is wholly satisfactory for long-term implantation.

A further object of the present invention is to provide an improved implantable medical cuff with an electrode array with a more flexible structure, in order to minimize the possibility of neural damage.

### SUMMARY OF THE INVENTION

The invention comprises an implantable medical cuff with an electrode array carried on a web of biocompatible film.

During surgical implantation, the softness and pliability of the electrode array enables it to be gently wound around a portion of body tissue with minimal manipulation of the tissue and minimal constriction of blood vessels. The implanted cuff completely encircles the nerve or other body tissue, to ensure proper communication with the body tissue, such as sub-bundles within the main nerve bundle. Once the cuff is implanted, the resiliency of the array and connecting cable effectively insulate the nerve from mechanical loads during body and muscle movement.

In one aspect, the invention comprises an electrode array having at least one electrode carried on a web of biocompatible film, the film having a plurality of slits dispersed throughout the web. The film may be shaped into a tube having a bore containing the electrode or electrodes on an inside surface of the bore and a longitudinal slit to allow the tube to encircle a nerve or nerve bundle. When the film is rolled into a tube, and placed around a portion of body tissue, the longitudinal edges of the film overlap, effectively sealing the tube and electrically isolating the tissue located within the tube.

In another aspect, the invention comprises at least one electrode, at least one undulated wire connected to the electrode to provide a stimulation signal, a biocompatible film within which the electrode and the wire are embedded, and at least one aperture provided on the biocompatible film.

The number of individual electrodes in the assembly is dictated by the specific form of neurostimulation to be achieved, but the assembly may comprise either single or multiple electrodes.

The invention also comprises various biomedical applications for different embodiments of the electrode array. The electrode array may be either implanted or attached to the skin. An electrode array may be employed for measuring the voltage potential of individual cells or of the surface area of an organ. However, in the preferred application, the electrode array is surgically implanted for establishing long term electrical contact with multiple cellular elements of an internal organ or tissue. The implanted electrode array may either electrically stimulate individual cells within the target organ or may sense nervous impulses within individual cells. Under some circumstances, the electrode array may both sense and stimulate electrical activity. Also, the electrical activity may be amplified and/or analyzed. Finally, the stimuli may be electronically correlated with the activity of the target cells.

In another aspect, the invention comprises an implantable medical cuff comprising a web of biocompatible film wound into a generally tubular configuration, with at least one electrode carried on an inside surface of the web, and further comprising at least one aperture through the web. The electrode may be connected to a wire, to provide a stimulation signal.

In a more specific aspect, the aperture may comprise a plurality of slits. The plurality of slits may be distributed throughout the web. The aperture or slits may be made by laser cutting or any other suitable means.

In a further aspect, the web of biocompatible film has opposed longitudinal edges that overlap in the generally tubular configuration.

In yet a further aspect, the electrode may comprise a plurality of electrodes arranged in spaced relation about a bore defined by the generally tubular configuration of the film.

In another aspect, the invention comprises an implantable medical cuff wherein the medical cuff is a nerve cuff electrode.

In another aspect, the invention comprises an implantable medical cuff wherein the cuff is in place about a portion of body tissue and is adapted to receive electrical impulses from that body tissue.

In yet another aspect, the invention comprises an implantable medical cuff wherein the aperture or apertures in the film are adapted to monitor concentrations of particular components of biological fluids within a body.

In another aspect, the invention comprises an implantable medical cuff wherein the aperture or apertures in the film are adapted to selectively transmit a particular chemical to a portion of body tissue about which the cuff is wrapped.

The foregoing was intended as a broad summary only and of only some of the aspects of the invention. It was not intended to define the limits or requirements of the invention. Other aspects of the invention will be appreciated by reference to the detailed description of the preferred embodiment and to the claims.

### BRIEF DESCRIPTION OF THE DRAWINGS

The preferred and alternative embodiments of the invention will be described by references to the accompanying drawings, in which:

FIG. 1 is a perspective view of a medical cuff with an electrode array according to an embodiment of the invention;

FIG. 2A is a cross-sectional view of a medical cuff with an electrode array according to an embodiment of the invention;

FIG. 2B is a cross-sectional view of a medical cuff with an electrode array when applied to the nerve according to an embodiment of the invention;

FIG. 3A is a planar view of a film which can be used to make a medical cuff with an electrode array according to an embodiment of the invention;

FIG. 3B is a planar view of the film of FIG. 3A, with slits made throughout, according to an embodiment of the invention;

FIG. 3C is a planar view of the film of FIG. 3B, with external pressure applied to the ends of the film;

FIG. 4A is a perspective view of the film of FIG. 3A, rolled into a relatively inflexible tube according to an embodiment of the invention; and

FIG. 4B is a view of the film of FIG. 3B, rolled into a flexible tube according to an embodiment of the invention.

### DETAILED DESCRIPTION OF THE PREFERRED EMBODIMENT

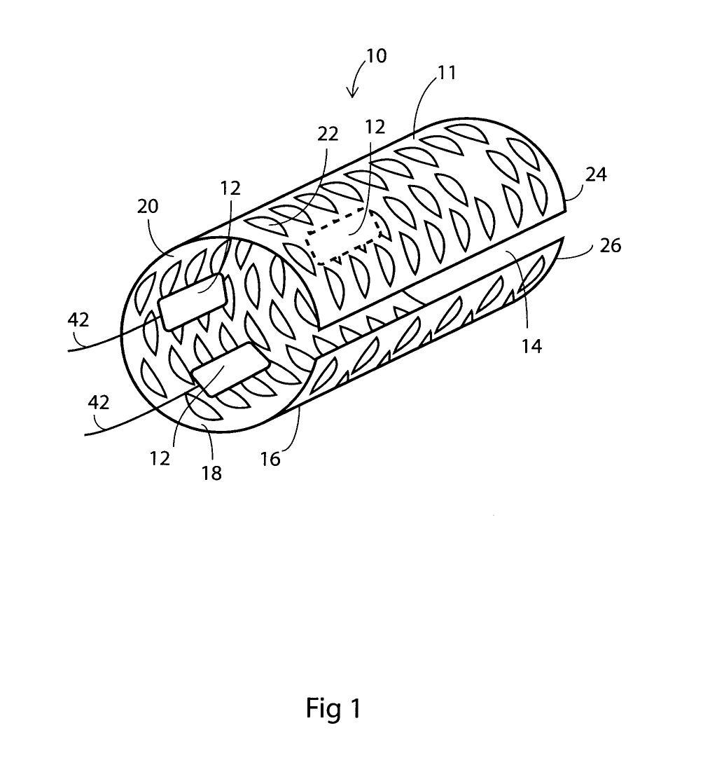

As shown in FIG. 1, a medical cuff 10 according to the present invention has a tubular cuff body 16 which has an inner surface 18 enclosing a generally cylindrical bore 20 for receiving a nerve or a portion of other body tissue (not shown). While this specification describes the cuff in association with a nerve, it is understood that the cuff is also adapted for use with other soft tissue, such as muscle. The cuff body 16 is formed from a web 11 of biocompatible film that is wound into a generally tubular configuration. Web 11 has opposed longitudinal edges 24, 26 allowing cuff body 16 to be opened, placed around an elongated portion of body tissue, and sealed with the tissue passing through the bore 20.

At least one aperture, such as slit 22, is established along the cuff body 16 through web 11 by any suitable method, such as laser cutting. Preferably, a plurality of such slits is distributed throughout web 11. The slits 22 allow fluids within the body to permeate the medical cuff 10, thereby providing proper nourishment into the tissue. That is, slits 22 effectively form fluid conduits or ducts between the wrapped portion of the tissue (e.g. the nerve bundle) and the surrounding biological fluids.

The cuff 10 includes at least one electrode 12 on the inside of the inner surface 18 of the web 11 of the cuff body 16. When the cuff body is placed around the nerve, each electrode is urged into contact with the wrapped tissue, without causing damage. Electrodes 12 may be used, for example, to selectively stimulate fascicles within a nerve (not shown in FIG. 1) passing through bore 20. The electrode array is a device for making multiple electrical contacts with cellular tissue or organs. Where a medical cuff is to be used for electrical stimulation, the material of the cuff body 16 should be electrically insulating.

Each of the electrodes 12 is individually connected through at least a single lead 42 to an operatively associated electrical generating source (not shown). Preferably, this wire is undulated to be more flexible and pliable against any force applied from the exterior.

Web 11 may be made of any suitable biocompatible material such as a biocompatible silicone. A fluoropolymer film may also be used to manufacture the cuff body 16. FEP or PFA film is used in the preferred embodiment according to the present invention. Further, it is possible to use other biomaterial such as fluorocarbons PVDF, PCTFF, ECTFE, ETFE, MFA (a copolymer of TFE and PVE), parelene-C, polyethylene's and polypropylenes. The thickness of the film may be about 20-100 pm. These materials are very flexible, resilient and electrically insulating.

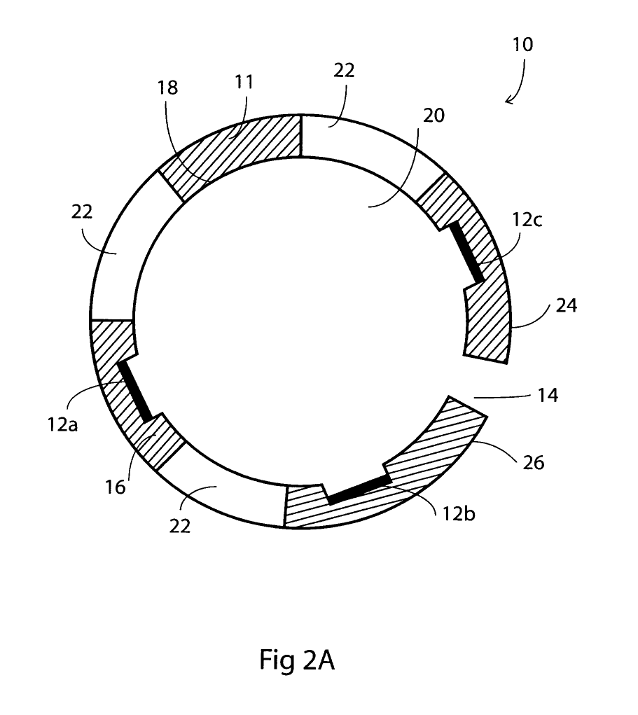

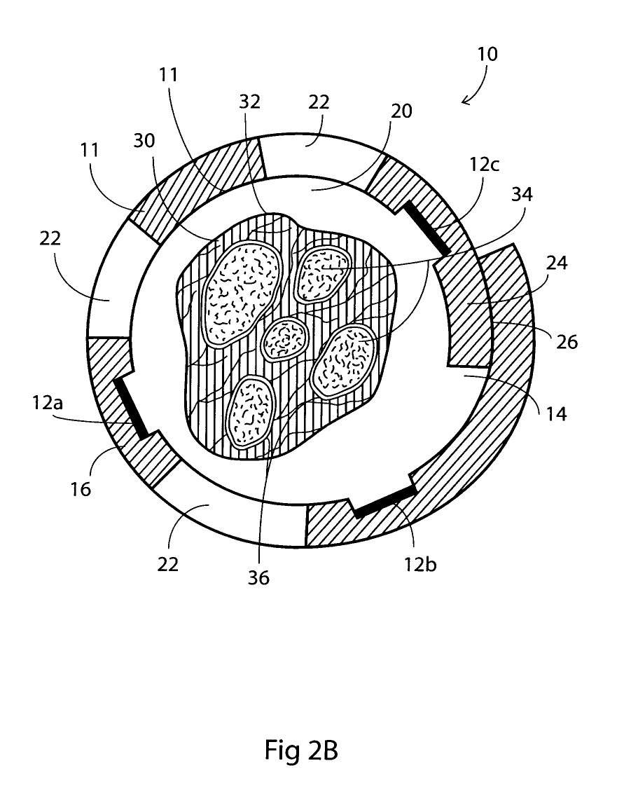

FIG. 2A shows a sectional view of a medical cuff 10 with an electrode array according to the present invention. The medical cuff 10 illustrated in FIG. 2A comprises a cuff body 16 having three electrodes 12a, 12b and 12c, in spaced relation about bore 20 along the length of the cuff body 16. The cuff body 16 may be electrically insulating as demanded by the application, and the electrodes 12a, 12b and 12c are made of a biologically compatible conductive material such as stainless steel, platinum, iridium or carbon. The cuff body 16 is dimensioned to fit loosely around a selected portion of body tissue, such as a nerve, in close proximity thereto, and has a length is preferably about ten times the inside diameter. It is to be understood that the electrode may be sized to fit the particular application and may be planar, multiplanar, curved, twisted, or otherwise shaped as desired to meet the requirements of the particular medical situation.

The cuff body 16 containing the circumferential electrodes 12a, 12b and 12c is slit longitudinally to permit the tube to be fit over the nerve (as will be discussed with reference to FIG. 2B); the longitudinal slit 14 is closed by overlapping the two opposing longitudinal edges of the cuff body 16, designated as flaps 24, 26, over the longitudinal slit 14, thus forming a generally tubular configuration.

Medical cuffs according to the present invention may be used to selectively record electrical signals or other electrical characteristics from portions of a nerve, to selectively electrically stimulate certain portions of a nerve, to selectively expose portions of a nerve to chemical or pharmacological agents or to selectively monitor the compositions of fluids surrounding certain portions of a nerve.

One application for a medical cuff with an electrode array is an implantable nerve cuff electrode, which is illustrated in FIG. 2B. This type of electrode surrounds the selected nerve in close proximity thereto. Close proximity of the electrode of the nerve cell axons or fibers is necessary because the conduction properties of the extracellular medium quickly attenuate the desired signals within a short distance of the source.

In general, the nerve 30 is surrounded by a loose membrane called the epineurium membrane 32. The nerve is typically organized into several groups of axons called fascicles 34. Each fascicle 34 is surrounded by a membrane called the perineurium membrane 36.

The dimensions of a medical cuff according to the invention will vary depending upon the size of the nerve to which the medical cuff will be applied. The cuff should be dimensioned so that the cuff body 16 gently but tightly sealed about the outer diameter of the nerve or nerve bundle. Instead of, or in addition to, making electrical contact with a nerve, the medical cuff according to the invention could be used to selectively expose portions of a nerve to pharmacological agents or other chemicals or to selectively sample fluids adjacent to portions of the surface of a nerve.

The electrical signals which stimulate the muscles normally have a prescribed frequency, pulse width and amplitude (typically a few milliamperes). These parameters are determined by conventional control circuitry included in the stimulator. This control circuitry is well known and described in the literature, and a number of different stimulators containing such control circuitry are similarly described. The primary input to this control circuitry is the control signal produced at the output of the summing junction.

The stimulator (not shown) can activate the desired muscles either by stimulating the muscle fibers directly or by stimulating the motor nerves which in turn control muscle activation. While the preferred stimulator uses implanted electrodes, i.e., electrodes implanted in the muscles or on selected muscle-controlling nerves, the present invention is not limited to the use of such stimulators. The present invention is also useful in FES systems using surface electrodes or percutaneous intramuscular electrodes as the stimulators. A variety of different stimulator electrodes are known in the literature. Recently proposed stimulators transmit the control signals to implanted electrodes by radio frequency so as to avoid the need for percutaneous connectors.

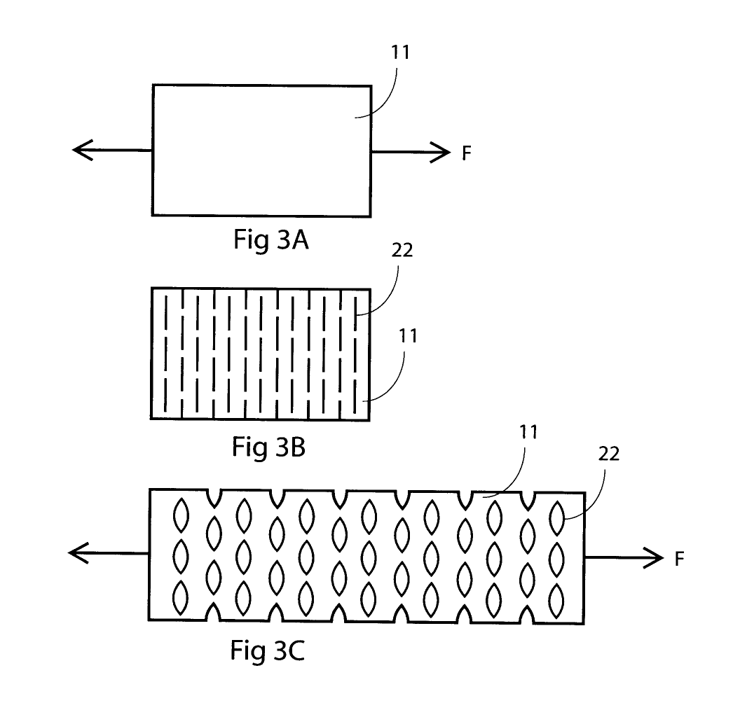

FIGS. 3A-3C show a planar view of a film which can be formed into a medical cuff according to the present invention. A continuous (i.e. without apertures) film is relatively stiff and unstretchable under application of external force F, as shown in FIG. 3A. Once multiple apertures in the form of slits (shown in FIG. 3B) are established on the film by any suitable method, such as laser cutting or any other mechanical or chemical method, the film becomes stretchable under external force F, as shown in FIG. 3C.

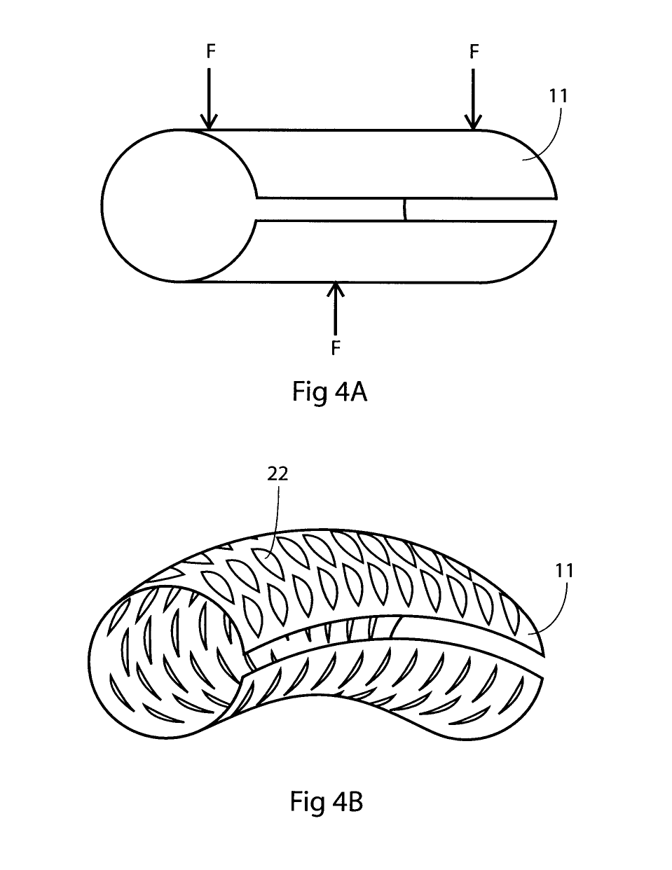

It will be appreciated that a medical cuff having a plurality of slits will also exhibit better mechanical flexibility once it is implanted around a tubular portion of biological tissue, when it is rolled into a tubular shape as shown in FIG. 4B, than will a similar tube without slits, which is shown in FIG. 4A. Those skilled in the art will appreciate that medical cuffs according to this invention can also allow the permeation of fluid from the exterior of the cuff. This property can be used to selectively allow biological fluids to access the enclosed tissue, to selectively transmit a particular chemical to the portion of body tissue about which the cuff is wrapped or to monitor the concentration of particular components of surrounding biological fluids within a body.

In view of the above discussion, it may be understood that the medical cuff of the present invention may provide stimulation to a group of muscles or successive stimulation to groups or portions of a group in order to achieve a desired muscular coordination. Such a medical cuff may be applied directly to or in the muscle, to or in selected nerves, or the central or peripheral nervous system to provide signals to the desired area. Also, a number of such electrode arrays may be applied at different locations and their stimulation or sensing coordinated to achieve desired results.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

1. An implantable medical cuff comprising;
a cuff body comprising a web of biocompatible film wound into a generally tubular configuration having opposed longitudinal edges and a substantially circular cross-section;
at least one electrode carried on an inside surface of said web; and
a plurality of distinct apertures distributed throughout said cuff body.

2. The implantable medical cuff of claim 1 wherein said plurality of distinct apertures comprise slits.

3. (canceled)

4. The implantable medical cuff of claim 1 wherein said opposed longitudinal edges overlap in said generally tubular configuration.

5. The implantable medical cuff of claim 1 wherein said at least one electrode comprises a plurality of electrodes arranged in spaced relation about a bore defined by said generally tubular configuration.

6. The implantable medical cuff of claim 1 wherein said medical cuff is a nerve cuff electrode.

7. The implantable medical cuff of claim 1 adapted to encircle a portion of body tissue wherein said medical cuff is adapted to receive electrical impulses from said body tissue.

8. The implantable medical cuff of claim 1 wherein said plurality of apertures is adapted to monitor concentrations of selected components of biological fluids within a body.

9. The implantable medical cuff of claim 1 wherein said plurality of apertures is adapted to selectively transmit a selected chemical to a portion of body tissue about which said cuff is wrapped.

10. The implantable medical cuff of claim 1 further comprising at least one wire connected to said at least one electrode to provide a stimulation signal.

11. The implantable medical cuff of claim 1 wherein said plurality of apertures is made by laser cutting.

12. The implantable medical cuff of claim 1 wherein said plurality of distinct apertures comprises a matrix of apertures distributed throughout said cuff body.

13. The implantable medical cuff of claim 11 wherein said apertures comprise slits.

14. The implantable medical cuff of claim 11 wherein said opposed longitudinal edges overlap in said generally tubular configuration.

15. The implantable medical cuff of claim 11 wherein said at least one electrode comprises a plurality of electrodes arranged in spaced relation about a bore defined by said generally tubular configuration.

16. The implantable medical cuff of claim 11 wherein said medical cuff is a nerve cuff electrode.

17. The implantable medical cuff of claim 11 adapted to encircle a portion of body tissue wherein said medical cuff is adapted to receive electrical impulses from said body tissue.

18. The implantable medical cuff of claim 11 wherein said plurality of apertures is adapted to monitor concentrations of selected components of biological fluids within a body.

19. The implantable medical cuff of claim 11 wherein said plurality of apertures is adapted to selectively transmit a selected chemical to a portion of body tissue about which said cuff is wrapped.

20. The implantable medical cuff of claim 11 further comprising at least one wire connected to said at least one electrode to provide a stimulation signal.

21. The implantable medical cuff of claim 11 wherein said plurality of apertures is made by laser cutting.
